(19) United States
(12) Patent Application Publication
(10) Pub. No.: US 2026/0309672 A1
(43) Pub. Date: Oct. 8, 2026

(54) BIO-FIELD-EFFECT TRANSISTOR WITH ENHANCED TEMPORAL STABILITY

(71) Applicant: International Business Machines Corporation, Armonk, NY (US)

(72) Inventors: Dario Goldfarb, Lake Carmel, NY (US); Kuan Yu Hsieh, Danbury, CT (US); Sufi Zafar, Briarcliff Manor, NY (US)

(21) Appl. No.: 19/172,142

(22) Filed: Apr. 7, 2025

(51) Int. Cl. G01N 27/414; G01N 33/543

(52) U.S. Cl. CPC G01N 27/4145; G01N 33/54373; G01N 2610/00

(57) ABSTRACT

One or more systems, devices and/or methods of fabrication provided herein relate to forming a semiconductor device for biosensing with enhanced temporal stability. According to an embodiment, a semiconductor device is provided. In various embodiments, the semiconductor device can comprise a semiconductor channel. In various embodiments, the semiconductor device can further comprise a gate electrode that can modulate current through the semiconductor channel, wherein the gate electrode can comprise two or more stacked layers, wherein the two or more stacked layers can be composed of an oxide or a nitride. In various embodiments, the semiconductor device can further comprise a biorecognition layer grafted to a top surface of the gate electrode.

### BACKGROUND

The subject disclosure relates to semiconductors and, more specifically, to bio-field-effect (bioFET) transistors with enhanced temporal stability.

### SUMMARY

The following presents a summary to provide a basic understanding of one or more embodiments described herein. This summary is not intended to identify key or critical elements, delineate scope of particular embodiments or scope of claims. Its sole purpose is to present concepts in a simplified form as a prelude to the more detailed description that is presented later. In one or more embodiments described herein, systems, computer-implemented methods, apparatus and/or computer program products that enable forming a semiconductor device for biosensing with enhanced temporal stability are discussed.

According to an embodiment, a semiconductor device is provided. In various embodiments, the semiconductor device can comprise a semiconductor channel. In various embodiments, the semiconductor device can further comprise a gate electrode that modulates current through the semiconductor channel, the gate electrode comprising two or more stacked layers, wherein the two or more stacked layers are composed of an oxide or a nitride. In various embodiments, the semiconductor device can further comprise a biorecognition layer grafted to a top surface of the gate electrode.

According to another embodiment, a method for fabricating a semiconductor device is provided. The method can comprise forming a semiconductor channel. The method can further comprise applying a gate electrode that modulates current through the semiconductor channel, the gate electrode comprising two or more stacked layers, wherein the two or more stacked layers are composed of an oxide or a nitride. The method can further comprise grafting a biorecognition layer to a top surface of the gate electrode.

According to another embodiment, a semiconductor device is provided. The semiconductor device can comprise a semiconductor channel, comprising a Silicon (Si) substrate. The semiconductor device can further comprise a gate electrode comprising a first layer stacked on a second layer, wherein the first layer or the second layer is a Group IV metal oxide or a Group IV metal nitride. The semiconductor device can further comprise a biorecognition layer grafted to a top surface of the gate electrode.

### BRIEF DESCRIPTION OF THE DRAWINGS

FIG. 1 illustrates an example, non-limiting cross-sectional view of a semiconductor device for biosensing with enhanced temporal stability in accordance with one or more embodiments described herein.

FIG. 2 illustrates an example, non-limiting graph of biosensor electrical signals using existing semiconductor devices in accordance with one or more embodiments described herein.

FIG. 3 illustrates an example, non-limiting cross-sectional view of a semiconductor device for biosensing with enhanced temporal stability in accordance with one or more embodiments described herein.

FIG. 4 illustrates an example, non-limiting diagram showing formation of a semiconductor device for biosensing with enhanced temporal stability in accordance with one or more embodiments described herein.

FIG. 5 illustrates an example, non-limiting diagram showing varied formations of a semiconductor device for biosensing with enhanced temporal stability in accordance with one or more embodiments described herein.

FIG. 6 illustrates an example, non-limiting diagram showing formation of a gate electrode with two layers in accordance with one or more embodiments described herein.

FIG. 7 illustrates example, non-limiting diagrams showing formation of a second layer of a gate electrode over a first layer of the gate electrode in accordance with one or more embodiments described herein.

FIG. 8 illustrates example, non-limiting experimental results showing temporal stability of a semiconductor device for biosensing comprising two or more stacked layers of a gate electrode in accordance with one or more embodiments described herein.

FIG. 9 illustrates example, non-limiting diagrams of possible materials to form a biorecognition layer and a biosensing agent in accordance with one or more embodiments described herein.

FIG. 10 illustrates an example, non-limiting energy-dispersive X-ray (EDX) mapping of a semiconductor device for biosensing with enhanced temporal stability in accordance with one or more embodiments described herein.

FIG. 11 illustrates an example, non-limiting bipolar junction transistor device comprising two or more stacked layers of a gate electrode in accordance with one or more embodiments described herein.

FIG. 12 illustrates an example, non-limiting field-effect transistor device comprising two or more stacked layers of a gate electrode in accordance with one or more embodiments described herein.

FIG. 13 illustrates a flow diagram of an example, non-limiting method in accordance with one or more embodiments described herein.

FIG. 14 illustrates a flow diagram of an example, non-limiting method in accordance with one or more embodiments described herein.

### DETAILED DESCRIPTION

The following detailed description is merely illustrative and is not intended to limit embodiments and/or application or uses of embodiments. Furthermore, there is no intention to be bound by any expressed or implied information presented in the preceding Background or Summary sections, or in the Detailed Description section.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

According to an embodiment, a semiconductor device is provided. In various embodiments, the semiconductor device can comprise a semiconductor channel. In various embodiments, the semiconductor device can further comprise a gate electrode that modulates current through the semiconductor channel, the gate electrode comprising two or more stacked layers, wherein the two or more stacked layers are composed of an oxide or a nitride. In various embodiments, the semiconductor device can further comprise a biorecognition layer grafted to a top surface of the gate electrode. Such embodiments can provide a number of advantages, including improving temporal stability of biosensing devices, reducing detachment of a biorecognition layer by preventing hydrolysis of the biorecognition layer, and improving biosensing accuracy.

In one or more embodiments of the semiconductor device, the oxide can be a Group IV metal oxide, and wherein the nitride can be a Group IV metal nitride. In one or more embodiments of the semiconductor device, the two or more stacked layers of the gate electrode can cause a reduction in desorption of the biorecognition layer into a solution. Such embodiments of the semiconductor device can provide the advantage of reducing detachment of a biorecognition layer by preventing hydrolysis of the biorecognition layer.

In one or more embodiments of the semiconductor device, the biorecognition layer can be an end-functionalized self-assembled monolayer. Such embodiment of the semiconductor device can provide a number of advantages, including improving sensitivity and performance of biosensors.

In one or more embodiments of the semiconductor device, the semiconductor device can further comprise a biosensing agent grafted to a top surface of the biorecognition layer. Such embodiment of the semiconductor device can provide a number of advantages, including enabling stronger interactions with a target analyte and thus more accurate sensor performance.

In one or more embodiments of the semiconductor device, the biosensing agent can be grafted to the top surface of the biorecognition layer via an interposed linker unit. Such embodiment of the semiconductor device can provide a number of advantages, including allowing for precise attachment of the biosensing agent to the biorecognition layer and improved temporal stability.

In one or more embodiments of the semiconductor device, the gate electrode can comprise a bulk material, wherein the bulk material is Hafnium dioxide (HfO₂) or Titanium nitride (TiN). In one or more embodiments of the semiconductor device, the gate electrode can further comprise a top surface material, wherein the top surface material is Zirconium dioxide (ZrO₂). Such embodiments of the semiconductor device can provide the advantage of reducing detachment of a biorecognition layer by preventing hydrolysis of the biorecognition layer.

In one or more embodiments of the semiconductor device, the semiconductor channel can comprise a Silicon (Si) substrate. In one or more embodiments of the semiconductor device, the semiconductor channel can comprise a Silicon Dioxide (SiO₂) substrate for optical biosensing. Such embodiment of the semiconductor device can provide a number of advantages, including enabling improved temporal stability for various types of biosensors and providing improved biosensing in various applications.

In one or more embodiments of the semiconductor device, the biorecognition layer can comprise alkylphosphonic acid with acidic (—COOH) or basic (—NH₂) end groups. Such embodiment of the semiconductor device can provide a number of advantages, including reducing detachment of a biorecognition layer and improving temporal stability of the biorecognition layer.

According to another embodiment, a method for fabricating a semiconductor device is provided. The method can comprise forming a semiconductor channel. The method can further comprise applying a gate electrode that modulates current through the semiconductor channel, the gate electrode comprising two or more stacked layers, wherein the two or more stacked layers are composed of an oxide or a nitride. The method can further comprise grafting a biorecognition layer to a top surface of the gate electrode.

According to another embodiment, a semiconductor device is provided. The semiconductor device can comprise a semiconductor channel, comprising a Silicon (Si) substrate. The semiconductor device can further comprise a gate electrode comprising a first layer stacked on a second layer, wherein the first layer or the second layer is a Group IV metal oxide or a Group IV metal nitride. The semiconductor device can further comprise a biorecognition layer grafted to a top surface of the gate electrode.

Such embodiments can provide a number of advantages, including improving temporal stability of biosensing devices, reducing detachment of a biorecognition layer by preventing hydrolysis of the biorecognition layer, and improving biosensing accuracy.

Biosensing is the process of detecting and measuring a target analyte in a test solution by using a sensor that converts a biological response into a measurable electrical signal for analysis. Various biosensing devices can include, but are not limited to, electrical biosensors (e.g., sensors that detect biological signals through electrochemical reactions), optical biosensors (e.g., sensors that use light to detect biological interactions), and optoelectronic biosensors (e.g., sensors that combine optical and electronic principles to convert biological responses into electrical signals).

In any case, a key advancement in biosensing is the use of semiconductor devices in biosensing devices. For instance, semiconductor biosensors are emerging as a leading choice for point-of care diagnostics (e.g., medical testing conducted at or near the site of patient care, enabling rapid results without the need for laboratory facilities). Semiconductor biosensors leverage semiconductor materials to convert biological interactions directly into electrical signals, which can enable faster response times and higher sensitivity. Various semiconductor biosensors can include, but are not limited to, field-effect transistors (FETs) and bipolar junction transistors (BJTs).

To detect a target analyte in a test solution, semiconductor biosensor devices comprise a gate electrode that is coated with a biorecognition layer post-fabrication. A gate electrode is a conductive component in a semiconductor device that modulates current through a semiconductor channel. In existing semiconductor devices, the gate electrode consists of a single conductive layer above or adjacent to the semiconductor channel. The biorecognition layer contains biomolecules that specifically interact with the target analyte (e.g., an ion, a protein, a virus), thus enabling the biorecognition layer to bind the target analyte. More specifically, when the biorecognition layer is exposed to the test solution, the target analyte binds to the biomolecules in the biorecognition layer, allowing the semiconductor biosensor to determine the amount of attachment by measuring the electrical signal produced.

However, when the biorecognition layer is exposed to the test solution, the biorecognition layer often detaches during measurement. Such problem occurs frequently in both electronic biosensors and optical biosensors. In either case, such temporal instability of the biorecognition layer causes a diminished sensor performance from the biosensing device.

First, detachment of the biorecognition layer causes sensitivity degradation of the semiconductor biosensor. That is, when the biorecognition layer detaches, the biosensor's ability to detect changes in the electric field or surface charge is impaired, leading to significant performance degradation and temporal instability. For example, in electrical biosensors, detachment of the biorecognition layer can result in diminished or absent electrical signals due to a loss in interaction between the biorecognition layer and target analyte. As another example, in optical biosensors, the detachment can disrupt the biosensor's ability to detect changes in light absorption caused by binding of the target analyte, leading to diminished or absent optical signals. In any case, the temporal instability of the biorecognition layer leads to sensitivity degradation of the biosensing device, as the loss or weakening of interaction with the target analyte results in inaccurate measurement and reduced performance over time.

Second, such temporal instability of the biorecognition layer induces detection limit degradation. As the biorecognition layer becomes unstable over time and detaches, its ability to consistently and specifically interact with the target analyte diminishes. This leads to a reduced sensitivity in the biosensing device as time progresses. In other words, the loss in binding of the target analyte by the biorecognition layer causes a degraded detection limit, resulting in weaker signals and less reliable measurements.

Third, the temporal instability of the biorecognition layer causes signal drifts. In other words, the biosensing device can exhibit unintended changes in the output signal over time, even when the concentration of the target analyte remains constant. Signal drifts can reduce measurement accuracy and reliability, especially in long-term monitoring applications. Thus, in addition to a detection limit degradation and sensitivity degradation, signal drifts further diminish the overall sensor performance of the biosensing device.

Thus, methods and structures that can address one or more of the challenges discussed herein can be desirable.

To that end, various embodiments herein relate to a unique structure and method of forming a semiconductor device for biosensing that can provide a number of advantages. For example, the various embodiments herein can comprise a semiconductor device that can be fabricated to comprise a semiconductor channel. The semiconductor device can further comprise a gate electrode that can modulate current through the semiconductor channel. In various embodiments, the gate electrode can comprise two or more stacked layers, wherein the two or more stacked layers can be composed of an oxide or a nitride. The semiconductor device can further comprise a biorecognition layer grafted to a top surface of the gate electrode.

The biorecognition layer can bind a target analyte in a test solution for biosensing. The two or more stacked layers of the gate electrode, as opposed to a single layer in existing methods, can reduce the detachment of the biorecognition layer and thus enhance temporal stability of the semiconductor device. More specifically, by comprising two or more stacked layers, the gate electrode can reduce desorption of the biorecognition layer when incubated in the test solution during detection of the target analyte. Therefore, the two or more stacked layers of the gate electrode can improve sensing signal stability and accuracy. That is, the various embodiments herein can reduce sensitivity degradation, detection limit degradation, and signal drifts in the semiconductor device for biosensing.

The various embodiments of the semiconductor device discussed herein can be applicable to electronic devices, such as high performance or low power logic devices, memories, etc.

The systems and/or devices have been (and/or will be further) described herein with respect to interaction between one or more components. Such systems and/or components can include those components or sub-components specified therein, one or more of the specified components and/or sub-components, and/or additional components. Sub-components can be implemented as components communicatively coupled to other components rather than included within parent components. One or more components and/or sub-components can be combined into a single component providing aggregate functionality. The components can interact with one or more other components not specifically described herein for the sake of brevity, but known by those of skill in the art.

One or more embodiments described herein can employ hardware and/or software to solve problems that are highly technical, that are not abstract, and that cannot be performed as a set of mental acts by a human. For example, a human, or even thousands of humans, cannot efficiently, accurately and/or effectively form a semiconductor channel in a semiconductor device as the one or more embodiments described herein can enable this process. And, neither can the human mind nor a human with pen and paper form a gate electrode comprising two or more stacked layers in a semiconductor device, as conducted by one or more embodiments described herein.

It should also be understood that when an element such as an interface layer, etc. is referred to as being “on” or “over” another element, it can be directly on the other element or intervening elements can also be present. In contrast, when an element is referred to as being “directly on” or “directly over” another element, there are no intervening elements present. It should also be understood that when an element is referred to as being “connected” or “coupled” to another element, it can be directly connected or coupled to the other element or intervening elements can be present. In contrast, when an element is referred to as being “directly connected” or “directly coupled” to another element, there are no intervening elements present.

These and other aspects and embodiments of the disclosed subject matter will now be described with respect to the drawings. It is to be appreciated that the words “semiconductor device” and “biosensing device” have been used interchangeably throughout this specification.

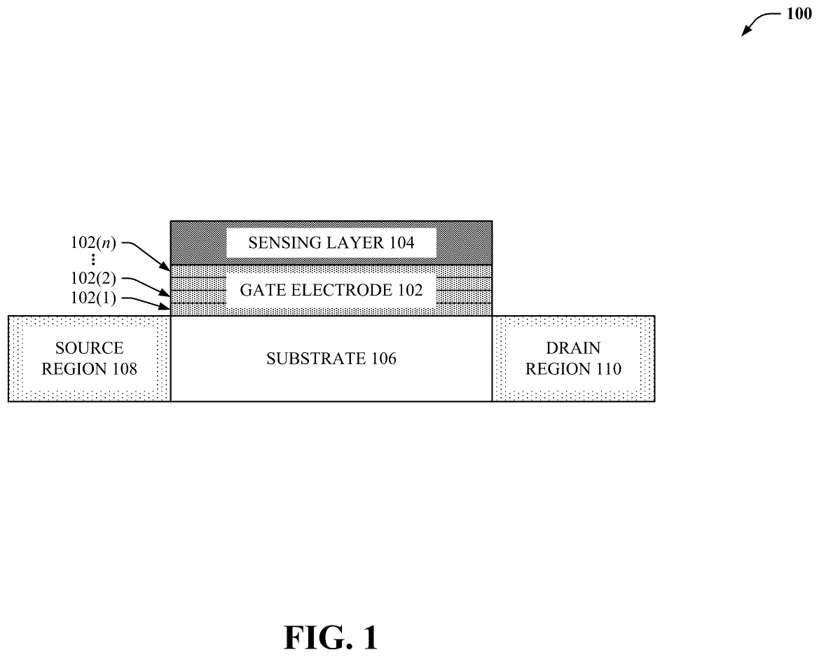

FIG. 1 illustrates an example, non-limiting cross-sectional view 100 of a semiconductor device for biosensing with enhanced temporal stability in accordance with one or more embodiments described herein

In an embodiment, a semiconductor device can comprise a semiconductor channel. In various embodiments, the semiconductor channel can comprise a substrate 106, a source region 108 and a drain region 110. The source region 108 and the drain region 110 can enable current to flow through the semiconductor channel. In various aspects, the source region 108 and the drain region 110 can be embedded into substrate 106 on opposite ends, as shown in FIG. 1.

In an embodiment, the semiconductor device can further comprise a gate electrode 102 that modulates the current through the semiconductor channel. In various embodiments, the gate electrode 102 can comprise two or more stacked layers: a layer 102(1) to a layer 102(n) for any positive integer n>1. Furthermore, the semiconductor device can comprise a sensing layer 104 above the gate electrode. In various aspects, the sensing layer 104 can interact with a target analyte in a test solution.

In embodiments described herein, the two or more stacked layers of gate electrode 102 can be composed of an oxide or a nitride. In various aspects, each of the two or more stacked layers can be composed of different materials. For instance, the layer 102(1) can be an oxide and layer 102(2) can be a nitride. In other words, the material from which each layer in the gate electrode is composed can differ between layers, so long as each layer is an oxide or a nitride. In various instances, the oxide can be a Group IV metal oxide, and the nitride can be a Group IV metal nitride.

It is to be appreciated that several layers and features of the semiconductor device illustrated in the cross-sectional views of FIG. 1 are also illustrated in cross-sectional views shown in other figures, although only some layers are discussed in detail for sake of brevity.

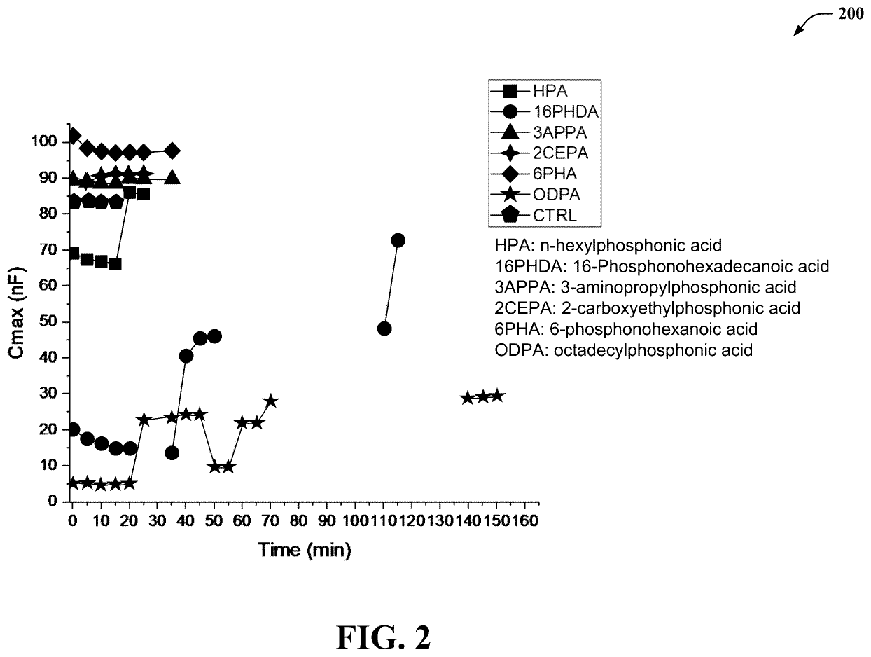

FIG. 2 illustrates an example, non-limiting graph 200 of biosensor electrical signals using existing semiconductor devices in accordance with one or more embodiments described herein.

Graph 200 depicts capacitance readout (denoted by Cₘₐₓ) over time for various biorecognition layers grafted onto existing biosensing devices, where the gate electrode comprises only a single layer. Biosensing devices based on the surface detection of biologically active agents through electrical and optical measurements depend on the chemical stability of the sensor surface immersed in aqueous media over time. Specifically, chemical bonding at the sensor surface should be resistant to hydrolysis (e.g., the chemical breakdown of a molecular structure due to its reaction with water) within a biologically relevant pH range to ensure stable capacitance readout.

As shown, when using existing biosensing devices with single layer gate electrode, the capacitance readout (e.g., signal) across the different biorecognition layers exhibits large variability over time, indicating inconsistent chemical stability of the sensor surface over time. For example, 16-Phosphonohexadecanoic acid (16PHDA), represented by circles, and octadecylphosphonic acid (ODPA), represented by stars, exhibit large fluctuations and variability in capacitance readout over time. This is due to hydrolysis of the biorecognition layers, weakening the bonds anchoring the biorecognition layer to the gate electrode and thus causing partial or complete detachment. As a result, the sensor surface becomes unstable, leading to fluctuations in capacitance readout and thus diminished sensor performance. Therefore, an improved linkage to the electrically active sensor surface is desirable. Accordingly, the various embodiments described herein can enable an improved linkage to the sensor surface (e.g., sensing layer 104) by depositing two or more stacked layers of a metal oxide or metal nitride in the gate electrode (gate electrode 102) and grafting the biorecognition layer to a top surface of the two or more stacked layers.

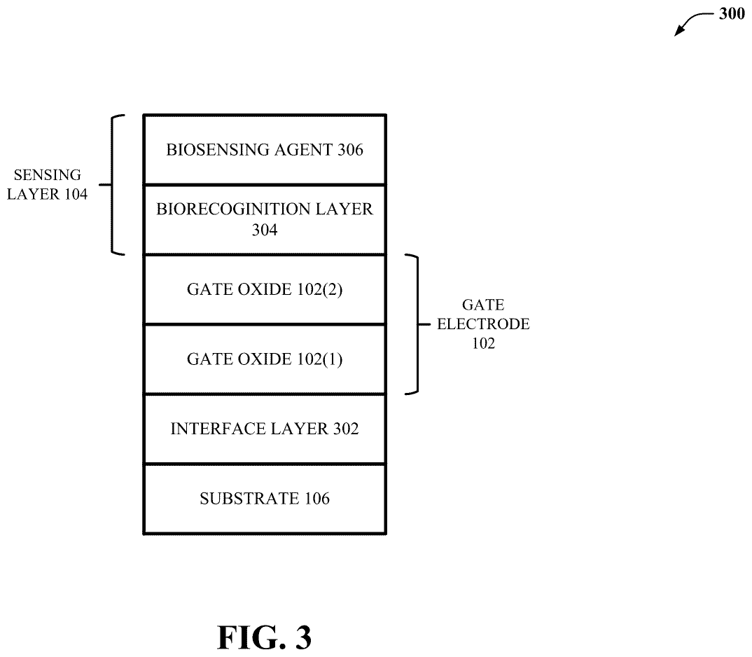

FIG. 3 illustrates an example, non-limiting cross-sectional view 300 of a semiconductor device for biosensing with enhanced temporal stability in accordance with one or more embodiments described herein. Repetitive description of like elements and/or processes employed in respective embodiments is omitted for sake of brevity.

In various embodiments, the semiconductor device can comprise substrate 106. As a non-limiting example, the substrate 106 can be Silicon (Si). However, the substrate 106 can be formed from any suitable material or elements. In various embodiments, the semiconductor device can comprise an interface layer 302 between the substrate 106 and the gate electrode 102. In various aspects, the interface layer 302 can be composed of any suitable dialectic material, such as but not limited to Silicon Dixide (SiO₂), Silicon Oxynitride (SiON), or Aluminum Oxide (Al₂O₃). In any case, the interface layer 302 can facilitate interaction between the gate electrode 102 and the substrate 106 (e.g., electrically isolate the gate electrode 102 from the substrate 106, modulate the electric field in the semiconductor channel when a surface charge is transferred to the gate electrode from the sensing layer 104).

In various embodiments, the gate electrode 102 can be positioned on top of the interface layer 302. Specifically, the gate electrode 102 can comprise two or more stacked layers. In various aspects, the two or more stacked layers of the gate electrode 102 can be composed of any suitable metal oxide or metal nitride. As a non-limiting example, as shown in FIG. 3, the gate electrode 102 can comprise two stacked layers (e.g., layer 102(1) and layer 102(2)), where layer 102(1) is composed of a metal oxide and layer 102(2) is composed of a metal oxide.

Note that this is a non-limiting example and that the gate electrode 102 can comprise more than two stacked layers. Additionally, note that although layer 102(1) and layer 102(2) are both described as being composed of metal oxide, layer 102(1) and layer 102(2) can be composed of different materials. For example, layer 102(1) can be composed of a metal nitride and layer 102(2) can be composed of a metal oxide. As another example, layer 102(1) can be composed of a metal oxide and layer 102(2) can be composed of a metal nitride. As still another example, layer 102(1) can be composed of a metal nitride and layer 102(2) can be composed of a metal nitride. In instances where the gate electrode 102 comprise more than two stacked layers, each of the more than two stacked layers can be composed of differing materials. For example, layer 102(1) and layer 102(2) can be composed of a metal oxide and layer 102(3) can be composed of a metal nitride. As another example, layer 102(1) can be composed of a metal oxide and layer 102(2) and layer 102(3) can be composed of a metal nitride. As still another example, layer 102(1) and layer 102(3) can be composed of a metal nitride and layer 102(2) can be composed of a metal oxide.

In various embodiments, the sensing layer 104 can comprise a biorecognition layer 304. In various aspects, the biorecognition layer 304 can be grafted to a top surface of the gate electrode 102. That is, the biorecognition layer 304 can be grafted to a top surface of layer 102(n) of the gate electrode 102.

Grafting is any process that covalently attaches molecules, such as polymers or biomolecules, onto a surface to modify its chemical or physical properties. Available techniques include, but are not limited to, surface-initiated polymerization, silanization, thiol-gold bonding, carbodiimide coupling, or click chemistry, among others.

In various aspects, the biorecognition layer 304 can be a self-assembled monolayer (SAM). A SAM is an organized, single-molecule-thick layer that forms on a solid surface through the chemisorption of functional molecules. In various aspects, the biorecognition layer 304 can be composed of any suitable biomolecule or surface modification material, such as but not limited to, antibodies, enzymes, aptamers, or proteins. In any case, the biorecognition layer 304 can bind a target analyte in a test solution. That is, the biorecognition layer 304 can facilitate the selective interaction between the semiconductor device and the target analyte, enabling the semiconductor device to detect specific biological or chemical species.

In various embodiments, the sensing layer 104 can further comprise a biosensing agent 306. In various aspects, the biosensing agent 306 can have biosensing capabilities. In various aspects, the biosensing agent 306 can be grafted to a top surface of the biorecognition layer 304. A biosensing agent is a biological or synthetic molecule that specifically interacts with a target analyte to enable detection of the target analyte in a biosensing device. In various instances, the biosensing agent 306 can be composed of any suitable chemical compound or biological material, such as but not limited to, receptors, peptides, small molecules, or molecularly imprinted polymers.

In any case, when the target analyte binds to biomolecules in the biorecognition layer 304, it can induce a change in surface charge in the sensing layer 104. The change in surface charge is then transferred to the gate electrode 102, modulating the electric field in the semiconductor channel and producing a measurable signal that correlates with the presence and concentration of the target analyte.

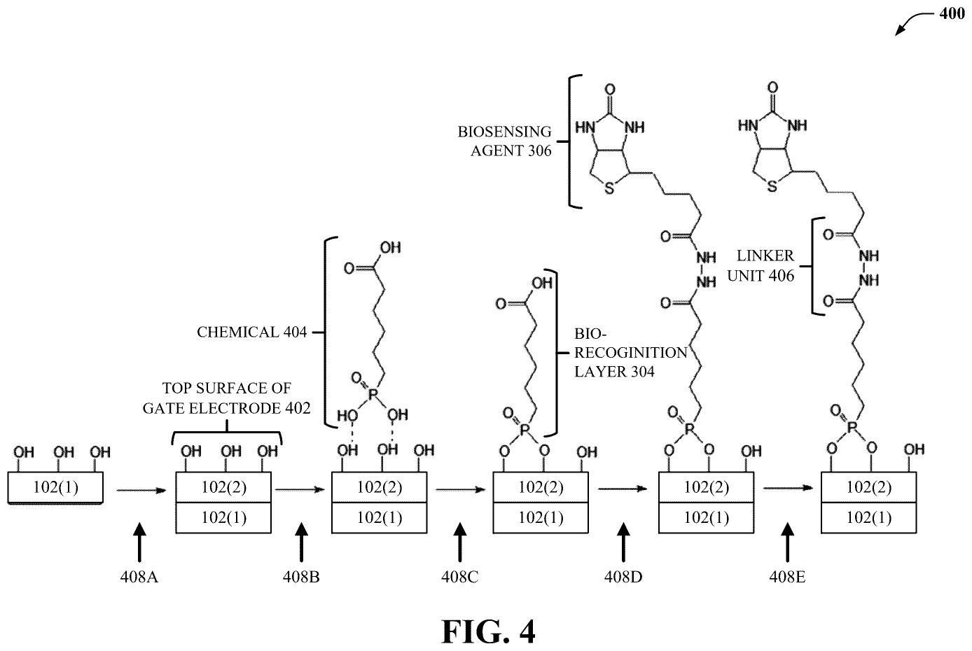

FIG. 4 illustrates an example, non-limiting diagram 400 showing formation of a semiconductor device for biosensing with enhanced temporal stability in accordance with one or more embodiments described herein. Repetitive description of like elements and/or processes employed in respective embodiments is omitted for sake of brevity.

As shown in the FIG. 4, non-limiting steps to form the semiconductor device for biosensing are denoted by step 408A, step 408B, step 408C, 408D, and 408E. In various aspects, at step 408A, layer 102(2) can be deposited on layer 102(1) to form gate electrode 102.

Deposition is any process that grows, coats, or otherwise transfers a material onto a substrate. Available technologies include, but are not limited to, dielectric spin-on, thermal oxidation, physical vapor deposition (PVD), chemical vapor deposition (CVD), electrochemical deposition (ECD), molecular beam epitaxy (MBE), or atomic layer deposition (ALD) among others.

Thereafter, at step 408B, chemical 404 can be added onto a top surface of gate electrode 402. Thus, through self-assembly at step 408C, the biorecognition layer 304 (e.g., SAM 204) can be formed as molecules of chemical 404 spontaneously organize into a single layer on the top surface of gate electrode 402. Specifically, the molecules of chemical 404 bind to the top surface of gate electrode 402, creating an organized monolayer that serves as the biorecognition layer 304.

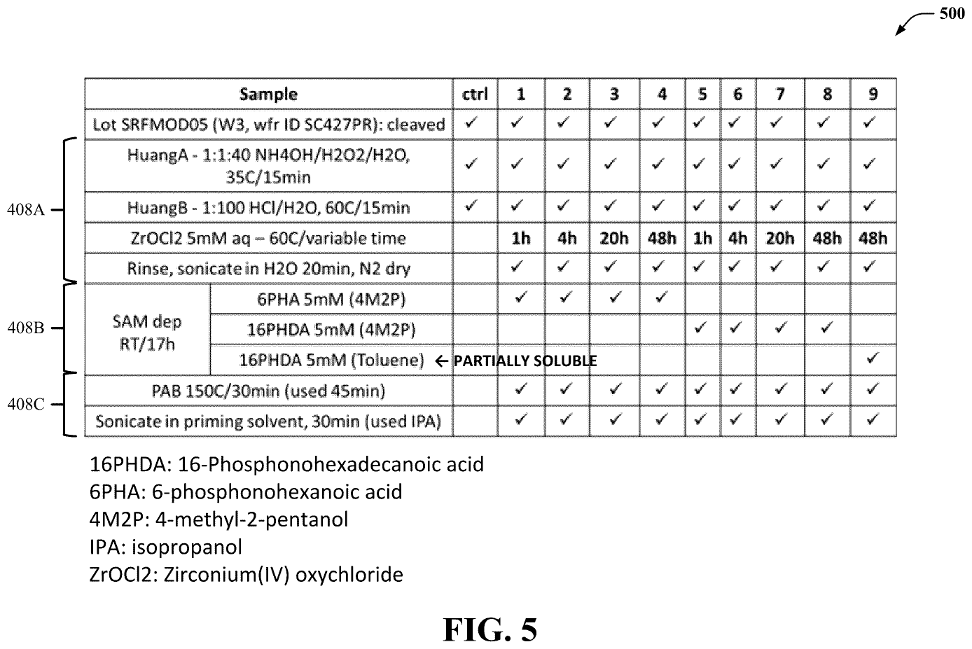

Following formation of the biorecognition layer 304, at step 408D, the biosensing agent 306 can be attached to the biorecognition layer 304. In various aspects, the biosensing agent 306 can be any suitable amino-terminated group. In various embodiments, a linker unit 406 can be used to attach the biosensing agent 306 to the biorecognition layer 304. In any case, at step 408E, Streptavidin attachment (SVD) can be performed on the biosensing agent 306 to test the biochemical sensitivity and temporal stability of the thereby formed biosensing multilayer assembly comprised of substrate 106, interface layer 302, gate electrode 102, biorecognition layer 304, and biosensing agent 306. Various non-limiting aspects of fabricating the semiconductor device for biosensing are described with respect to FIG. 5.

FIG. 5 illustrates an example, non-limiting diagram 500 showing varied formations of a semiconductor device for biosensing with enhanced temporal stability in accordance with one or more embodiments described herein. Repetitive description of like elements and/or processes employed in respective embodiments is omitted for sake of brevity.

In various aspects, non-limiting diagram 500 depicts 9 sample biosensing devices with two or more stacked layers of gate electrode 102 and a control biosensing device, where the 9 sample biosensing devices are fabricated with different materials. Specifically, samples 1-4 use 6PHA 5 mM (4M2P) to form the biorecognition layer 304, samples 5-8 use 16PHDA 5 mM (4M2P) to form the biorecognition layer 304, and sample 9 uses 16PHDA 5 mM (Toluene) to form the biorecognition layer 304, where 16PHDA 5 mM (Toluene) is partially soluble.

Referring to FIGS. 4 and 5, to form the semiconductor device for biosensing with two or more stacked layers of gate electrode 102, substrate 106 can first be cleaned. In various instances, additional cleanings of the substrate 106 can also be performed. In any case, a semiconductor wafer that forms the substrate 106 can already contain an oxide or nitride film and can thus serve as layer 102(1) of the gate electrode 102. In other words, the top surface of the substrate 106 can have oxide or nitride functionality.

Thereafter, at step 408A, layer 102(1) of the gate electrode 102 can be cleaned (e.g., cleaned in two steps). Following cleaning of layer 102(1) of gate electrode 102, a solution can be used to grow layer 102(2) of gate electrode 102. For example, in samples 1-9 as shown in diagram 500, a Zirconium oxychloride solution can be used to grow a Zirconium dioxide layer of the gate electrode 102.

Then, layer 102(2) of gate electrode 102 can be cleaned (e.g., rinsed and sonicated). Following cleaning of layer 102(2), the biorecognition layer 304 can be grafted to the top surface of the gate electrode 402. That is, at step 408B, chemical 404 can be deposited on the top surface of the gate electrode 402. Particularly, 6PHA 5 mM (4M2P) is deposited in samples 1-4, 16PHDA 5 mM (4M2P) is deposited in samples 5-8, and 16PHDA 5 mM (Toluene) is deposited in sample 9.

Thereafter, at step 408C, annealing or soaking a post applied bake can be performed. Annealing is any process that involves heating a material to a specific temperature and then cooling it in a controlled manner to alter its physical or chemical properties. Available techniques include, but are not limited to, rapid thermal annealing (RTA), furnace annealing, laser annealing, or spike annealing, among others. Step 408C can further involve sonicating the biorecognition layer 304 to remove excess material. Sonicating is any process that uses sound energy, typically ultrasonic waves, to agitate particles in a solution or clean surfaces. Available techniques include, but are not limited to, immersion sonication, probe sonication, and ultrasonic cleaning, among others.

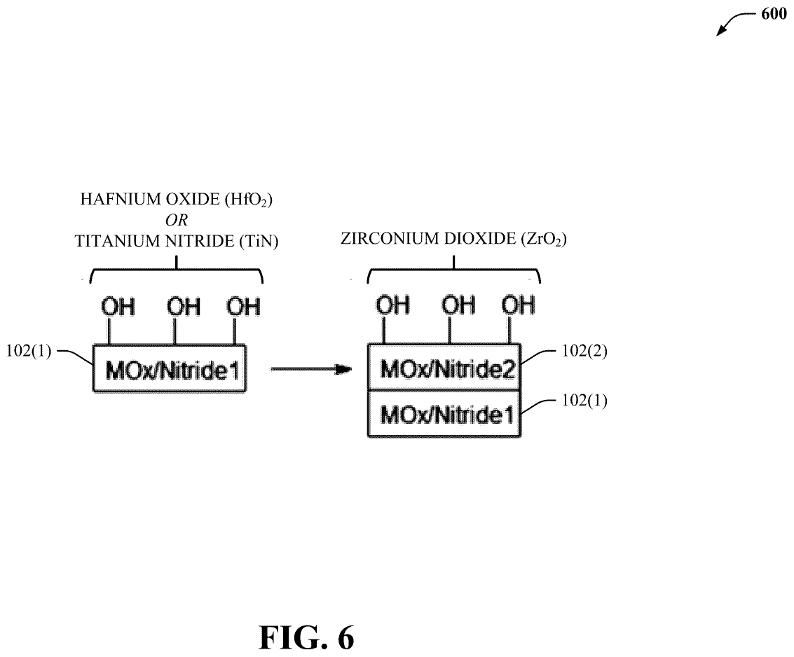

FIG. 6 illustrates an example, non-limiting diagram 600 showing formation of a gate electrode with two layers in accordance with one or more embodiments described herein. Repetitive description of like elements and/or processes employed in respective embodiments is omitted for sake of brevity.

Diagram 600 shows a non-limiting example of forming a stacked layer of the gate electrode 102 on another layer of the gate electrode 102. As a non-limiting example, layer 102(1) can be composed of Hafnium dioxide or Titanium nitride. Accordingly, layer 102(2) can be deposited on the Hafnium dioxide or Titanium nitride. As a non-limiting example, layer 102(1) can be composed of Zirconium dioxide.

In various aspects, the gate electrode 102 can comprise a bulk material and a top surface material. In various embodiments, the top surface material can be the material forming the top-most layer of gate electrode 102 (e.g., layer 102(n)). The bulk material can be the material forming any layer below the top-most layer of gate electrode 102 (e.g., layer 102(1) to layer 102(n-1)). In various instances, the bulk material can consist of different materials if the gate electrode 102 comprises more than two layers.

In the non-limiting example of FIG. 6, the Hafnium dioxide or Titanium nitride can be considered the bulk material, and the Zirconium dioxide can be considered the top surface material.

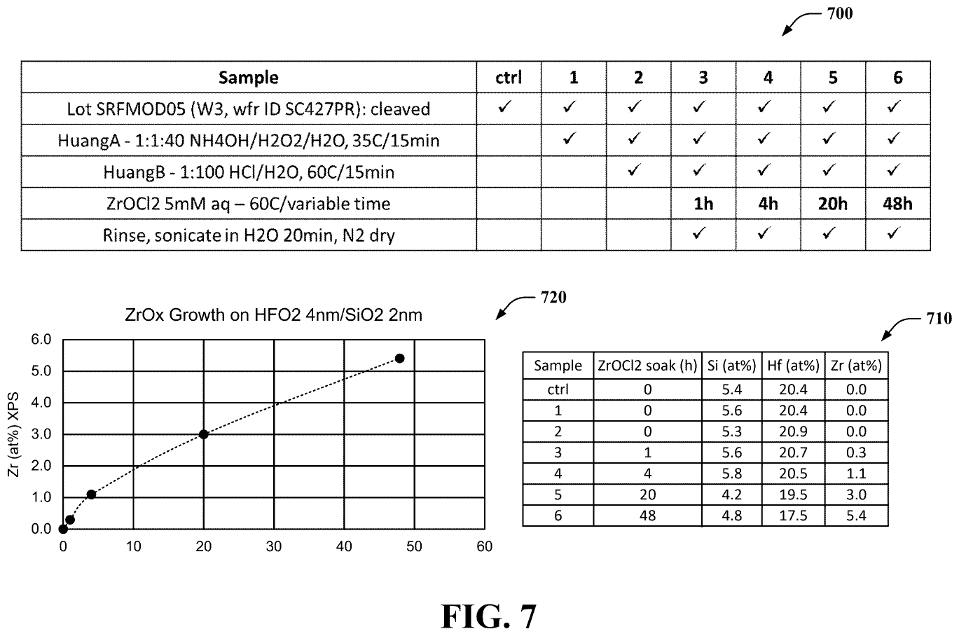

FIG. 7 illustrates example, non-limiting diagrams 700, 710, and 720 showing formation of a second layer of a gate electrode over a first layer of the gate electrode in accordance with one or more embodiments described herein. Repetitive description of like elements and/or processes employed in respective embodiments is omitted for sake of brevity.

Diagram 700 depicts a portion of diagram 500 showing the steps to form the two or more layers of the gate electrode 102. Diagram 720 depicts the signal of a Zirconium dioxide layer (layer 102(1)) of the gate electrode 102 as a function of time. As shown, the Zirconium dioxide layer exhibits monotonic growth using conditions defined in diagram 710.

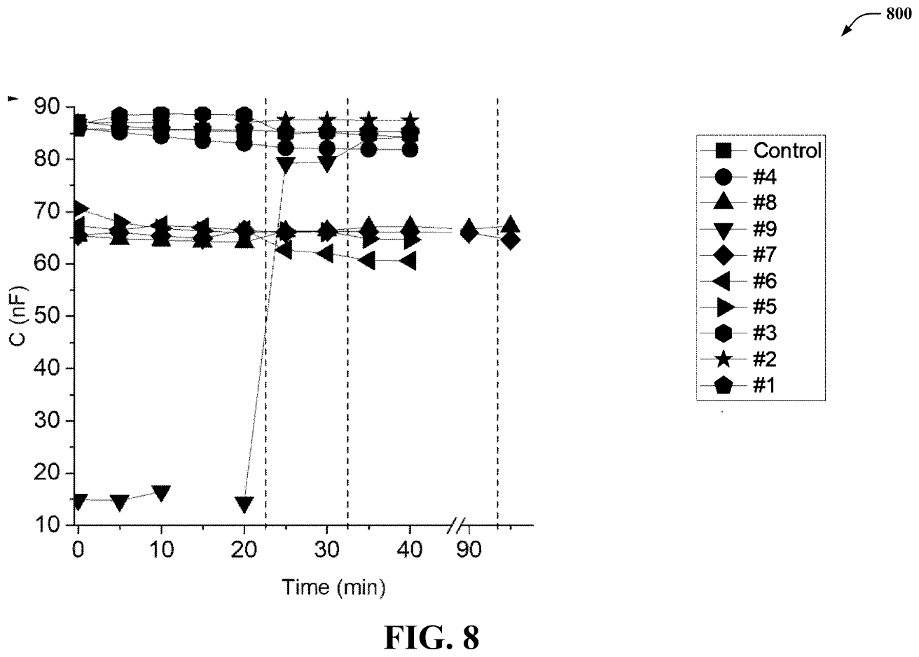

FIG. 8 illustrates example, non-limiting experimental results showing temporal stability of a semiconductor device for biosensing comprising two or more stacked layers of a gate electrode in accordance with one or more embodiments described herein. Repetitive description of like elements and/or processes employed in respective embodiments is omitted for sake of brevity.

Graph 800 depicts capacitance readout (denoted by C) over time for various biorecognition layers grafted onto a semiconductor device, where the gate electrode 102 comprises two layers. Specifically, graph 800 depicts capacitance readout for sample biosensing devices 1-9 and the control biosensing device. As shown, the capacitance readout for the various biorecognition layers when the semiconductor device comprises two or more stacked layers of gate electrode 102 exhibit a stable signal over time and improved temporal stability. Conversely, as shown in graph 200, the capacitance readout from semiconductor devices with a single layer of a gate electrode exhibits large variability over time and poor temporal stability. Thus, the capacitance readout is significantly more stable with reduced variability when the gate electrode 102 comprises two or more stacked layers. More specifically, the two or more stacked layers of the gate electrode 102 can cause a reduction in desorption of the biorecognition layer 304 into the test solution (e.g., prevents hydrolysis of the biorecognition layer 304). Therefore, as shown by graph 200 and graph 800, by depositing two or more stacked layers of the gate electrode 102 in biosensing devices, the biosensing devices can produce significantly more stable signals by preventing detachment of the biorecognition layer 304.

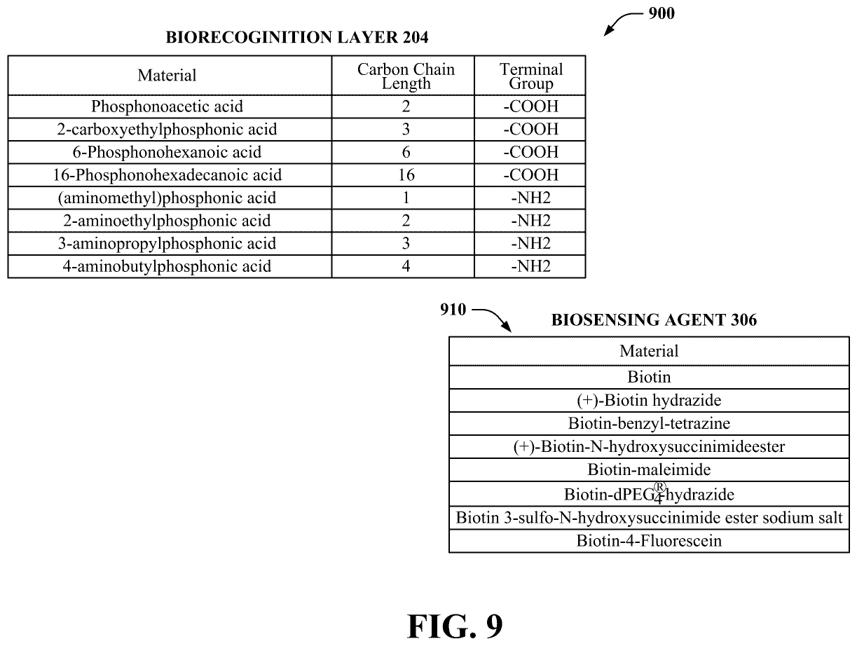

FIG. 9 illustrates example, non-limiting diagrams of possible materials to form a biorecognition layer and a biosensing agent in accordance with one or more embodiments described herein.

In various embodiments, the biorecognition layer 304 and the biosensing agent 306 can be composed of or formed from any suitable material or elements.

Table 900 depicts non-limiting examples of possible materials to form the biorecognition layer 304. In various cases, the biorecognition layer 304 can comprise acidic (—COOH) end groups (e.g., terminal groups). As shown in table 900, non-limiting examples of possible materials with acidic end groups to form the biorecognition layer 304 can include, but are not limited to, phosphonoacetic acid, 2-carboxyethylphosphonic acid, 6-phosphonohexanoic acid, or 16-phosphonohexadecanoic acid. In other instances, the biorecognition layer 304 can comprise basic (—NH₂) end groups. As shown in table 900, non-limiting examples of possible materials with basic end groups to form the biorecognition layer 304 can include, but are not limited to, (aminomethyl) phosphonic acid, 2-aminoethylphosphonic acid, 3-aminopropylphosphonic acid, or 4-aminobutylphosphonic acid.

Table 910 depicts non-limiting examples of possible materials to form the biosensing agent 306. In various aspects, the biosensing agent 306 can be composed of biotin. As shown in table 910, non-limiting examples of possible materials to form the biosensing agent 306 can include, but are not limited to, (+)-biotin hydrazide, biotin-benzyl-tetrazine, (+)-biotin N-hydroxysuccinimide ester, biotin-maleimide, biotin-dPEG®₄-hydrazide, biotin 3-sulfo-N-hydroxysuccinimide ester sodium salt, or biotin-4-fluorescein.

Note that these are non-limiting examples, and that the biorecognition layer 304 and the biosensing agent 306 can be composed of or formed from any suitable material or elements.

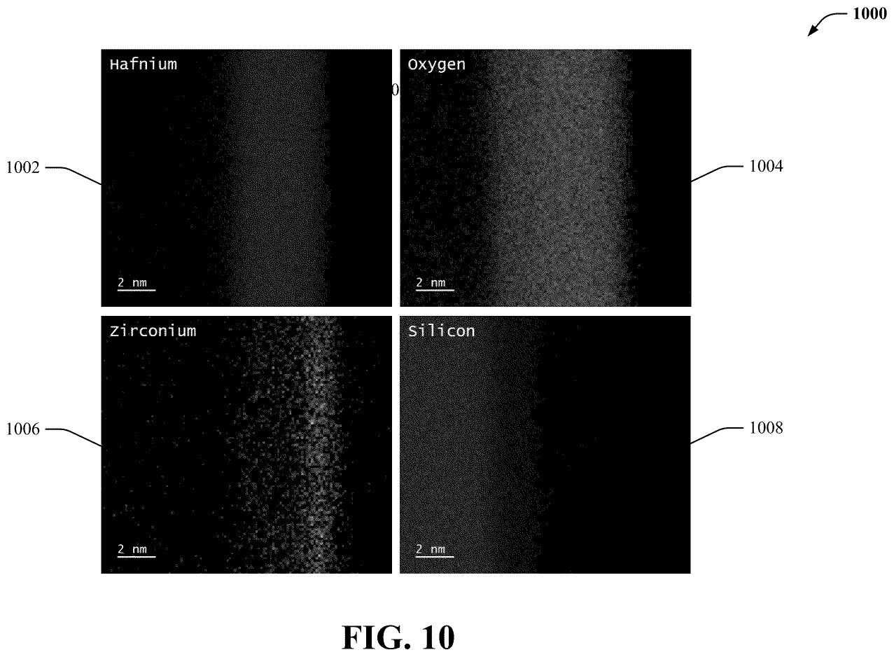

FIG. 10 illustrates an example, non-limiting energy-dispersive X-ray (EDX) mapping 1000 of a semiconductor device for biosensing with enhanced temporal stability in accordance with one or more embodiments described herein.

EDX mapping 1000 depicts the layers of the semiconductor device described herein. EDX mapping is a technique used in materials science and semiconductor analysis to visualize the elemental composition and distribution across a sample's surface or cross-section. EDX mapping 1000 consists of mapping 1002, mapping 1004, mapping 1006, and mapping 1008.

Mapping 1002 depicts layer 102(1) of the gate electrode 102, where layer 102(1) is composed of Hafnium. As shown, layer 102(1) of the gate electrode 102 can comprise a thickness of approximately 4 nm. Mapping 1006 depicts layer 102(2) of the gate electrode 102, where layer 102(2) is composed of Zirconium. As shown, layer 102(2) of the gate electrode 102 can be stacked on layer 102(1) and can comprise a thickness of approximately 1.5 nm. Mapping 1004 depicts Oxygen in the semiconductor device that transforms a Zirconium metal into the Zirconium dioxide, forming layer 102(2). Mapping 1008 depicts the substrate 106. As shown, substrate 106 can comprise a thickness of approximately 4 nm.

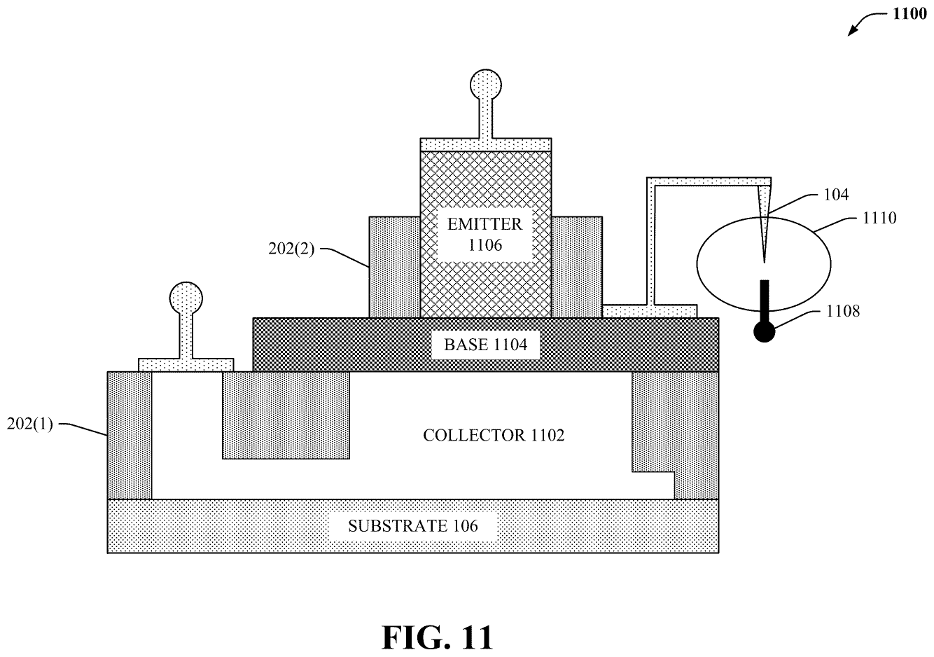

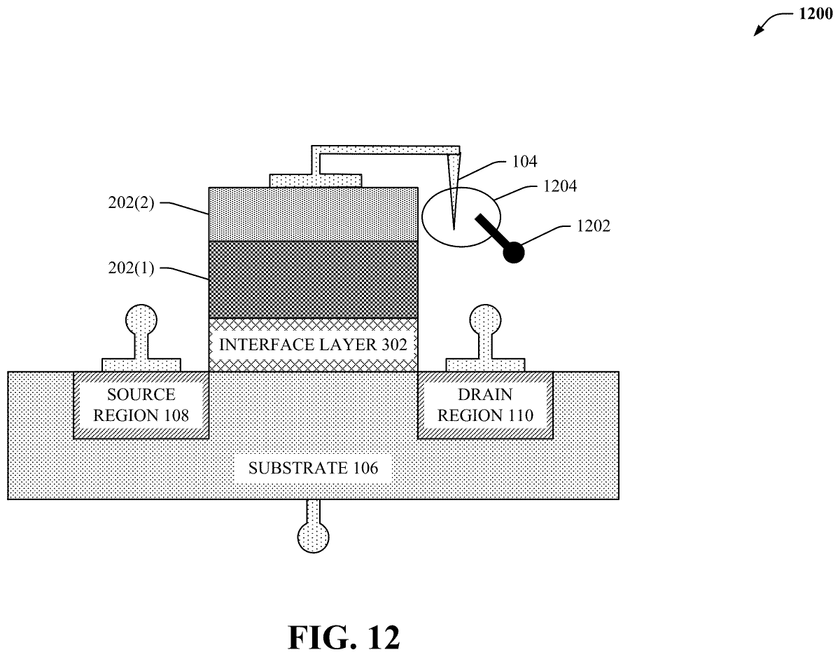

FIGS. 11 and 12 illustrate non-limiting examples of different types of semiconductor devices that can comprise two or more stacked layers of a gate electrode for biosensing.

FIG. 11 illustrates an example, non-limiting BJT device 1100 comprising two or more stacked layers of gate electrode 102 in accordance with one or more embodiments described herein.

In various aspects, the non-limiting BJT device 1100 can comprise substrate 106, two or more stacked layers of gate electrode 102, sensing layer 104 (e.g., sensing surface 104), a collector 1102, a base 1104, an emitter 1106, and a reference electrode 1108.

In various embodiments, the collector 1102 is a moderately doped region that can collect charge carriers passing through the base 1104. The collector 1102 can be positioned on the substrate and within layer 102(1) of the gate electrode 102.

The emitter 1106 is a heavily doped region that injects charge carriers into the base 1104. In various embodiments, the emitter 1106 can be positioned on the base 1104 and within layer 102(2) of the gate electrode 102.

The base 1104 is a thin, lightly doped layer that can be positioned between the collector 1102 and the emitter 1106. Accordingly, the base 1104 can control the flow of charge carriers from the emitter 1106 to the collector 1102.

The sensing surface 104 can interact with the target analyte of a test solution 1110. In various aspects, the sensing surface 104 can be positioned adjacent to the gate electrode 102. In some instances, the sensing surface 104 can be coated with Titanium nitride.

Furthermore, the reference electrode 1108 can provide a stable voltage reference for the non-limiting BJT device 1100. In various embodiments, the reference electrode 1108 can be positioned near the sensing surface 104 to maintain a fixed potential during sensing operations.

FIG. 12 illustrates an example, non-limiting FET device 1200 comprising two or more stacked layers of gate electrode 102 in accordance with one or more embodiments described herein.

In various aspects, the non-limiting FET device 1200 can comprise substrate 106, source region 108, drain region 110, two or more stacked layers of gate electrode 102, interface layer 302, sensing layer 104 (e.g., sensing surface 104), and a reference electrode 1202.

In various embodiments, the source region 108 is a terminal that can enable charge carriers to enter the semiconductor channel. In various embodiments, the source region 108 can be positioned on one side of the semiconductor channel and within the substrate 106.

The drain region 110 is a terminal that can enable charge carriers to exit the semiconductor channel. In various embodiments, the drain region 110 can be positioned on the opposite side of the semiconductor channel from the source region 108 and within the substrate 106.

The interface layer 302 is a dielectric material that can electrically isolate the gate electrode 102 from the semiconductor channel while enabling modulation of the electric field. In various embodiments, the interface layer 302 can be positioned between the substrate 106 and the gate electrode 102.

In various embodiments, the gate electrode 102 can comprise two or more stacked layers. In the non-limiting FET device 1200, the gate electrode 102 comprised two stacked layers. In any case, the two or more stacked layers of gate electrode 102 can control the flow of current between the source region 108 and the drain region 110.

The sensing surface 104 can interact with the target analyte of a test solution 1204. In various aspects, the sensing surface 104 can be positioned on the gate electrode 102. In some instances, the sensing surface 104 can be coated with Titanium nitride.

Furthermore, the reference electrode 1202 can provide a stable voltage reference for the non-limiting FET device 1200. In various embodiments, the reference electrode 1202 can be positioned near the sensing surface 104 to maintain a fixed potential during sensing operations.

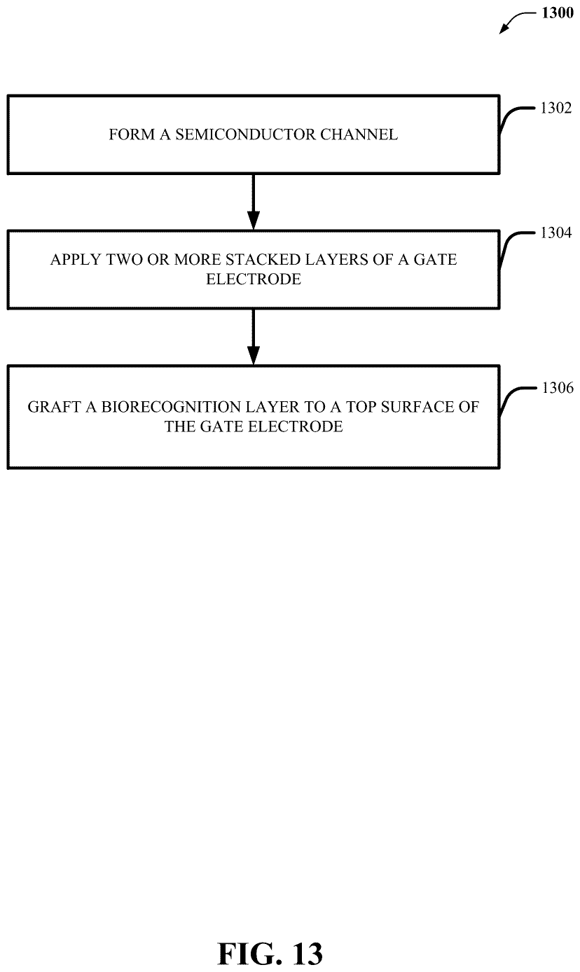

FIG. 13 illustrates a flow diagram of an example, non-limiting method 1300 in accordance with one or more embodiments described herein. Repetitive description of like elements and/or processes employed in respective embodiments is omitted for sake of brevity.

At 1302, the non-limiting method 1300 can comprise forming a semiconductor channel.

At 1304, the non-limiting method 1300 can comprise applying two or more stacked layers of a gate electrode (e.g., 102). Specifically, in various embodiments, the two or more stacked layers of gate electrode 102 can be formed by depositing each layer on a top surface of a previously deposited layer. For example, layer 102(2) can be deposited on a top surface of 102(1).

At 1306, the non-limiting method 1300 can comprise grafting a biorecognition layer (e.g., 304) to a top surface of the gate electrode (e.g., 402).

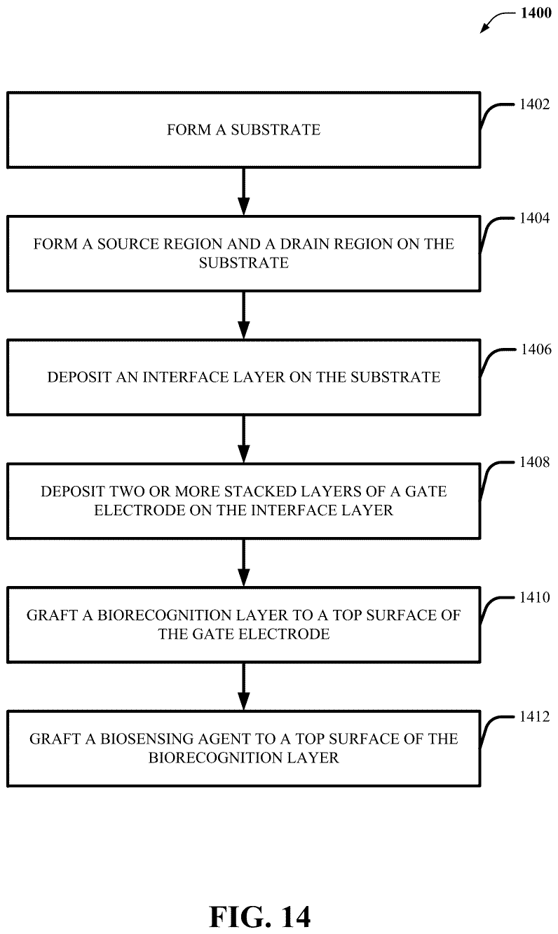

FIG. 14 illustrates a flow diagram of an example, non-limiting method 1400 in accordance with one or more embodiments described herein. Repetitive description of like elements and/or processes employed in respective embodiments is omitted for sake of brevity.

At 1402, the non-limiting method 1400 can comprise forming a substrate (e.g., 106).

At 1404, the non-limiting method 1400 can comprise forming a source region (e.g., 108) and a drain region (e.g., 110) on the substrate.

At 1406, the non-limiting method 1400 can comprise depositing an interface layer (e.g., 302) on the substrate.

At 1408, the non-limiting method 1400 can comprise depositing two or more stacked layers of a gate electrode (e.g., 102) on the interface layer.

At 1410, the non-limiting method 1400 can comprise grafting a biorecognition layer (e.g., 304) to a top surface of the gate electrode (e.g., 402).

At 1412, the non-limiting method 1400 can comprise grafting a biosensing agent (e.g., 306) to a top surface of the biorecognition layer.

In some embodiments, patterning can be performed after deposition of one or more layers of the semiconductor device (e.g., source region 108, drain region 110). Patterning is the shaping or altering of deposited materials, and is generally referred to as lithography. For example, in conventional lithography, the wafer is coated with a chemical called a photoresist; then, a machine called a stepper focuses, aligns, and moves a mask, exposing select portions of the wafer below to short wavelength light. The exposed regions are then washed away by a developer solution. After etching or other processing, the remaining photoresist is removed. After transferring the pattern, the patterned photoresist is removed utilizing resist stripping processes, such as wet chemical clean or ashing. Ashing can be used to remove a photoresist material, amorphous carbon, or organic planarization (OPL) layer. Ashing is performed using a suitable reaction gas, for example, O₂, N₂, H₂/N₂, O₃, CF₄, or any combination thereof. Patterning also includes electron-beam lithography, nanoimprint lithography, and reactive ion etching.

As an example, after the substrate 106 is deposited, patterning can be performed to define and create exposed areas that can be etched to define source region 108 and drain region 110.

Note that, although the semiconductor device described herein is primarily described as an FET electrical biosensor, the semiconductor device can be any suitable biosensing device comprising a gate electrode comprising two or more stacked layers of a metal oxide or metal nitride. For instance, the semiconductor device can be a BJT comprising a gate electrode comprising two or more stacked layers of a metal oxide or metal nitride. As another example, the semiconductor device can be an optical biosensor comprising a gate electrode comprising two or more stacked layers of a metal oxide or metal nitride. Particularly, the substrate 106 can be Silicon Dioxide (SiO₂) for optical biosensing.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

It is, of course, not possible to describe every conceivable combination of methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A semiconductor device, comprising:
a semiconductor channel;
a gate electrode that modulates current through the semiconductor channel, the gate electrode comprising two or more stacked layers, wherein the two or more stacked layers are composed of an oxide or a nitride; and
a biorecognition layer grafted to a top surface of the gate electrode.

2. The semiconductor device of claim 1, wherein the oxide is a Group IV metal oxide, and wherein the nitride is a Group IV metal nitride.

3. The semiconductor device of claim 1, wherein the two or more stacked layers of the gate electrode cause a reduction in desorption of the biorecognition layer into a solution.

4. The semiconductor device of claim 1, wherein the biorecognition layer is an end-functionalized self-assembled monolayer.

5. The semiconductor device of claim 1, further comprising:
a biosensing agent grafted to a top surface of the biorecognition layer.

6. The semiconductor device of claim 5, wherein the biosensing agent is grafted to the top surface of the biorecognition layer via an interposed linker unit.

7. The semiconductor device of claim 1, wherein the gate electrode comprises:
a bulk material, wherein the bulk material is Hafnium dioxide (HfO₂) or Titanium nitride (TiN); and
a top surface material, wherein the top surface material is Zirconium dioxide (ZrO₂).

8. The semiconductor device of claim 1, wherein the semiconductor channel comprises a Silicon (Si) substrate.

9. The semiconductor device of claim 1, wherein the semiconductor channel comprises a Silicon Dioxide (SiO2) substrate for optical biosensing.

10. The semiconductor device of claim 1, wherein the biorecognition layer comprises alkylphosphonic acid with acidic (—COOH) or basic (—NH₂) end groups.

11. A method, comprising steps of:
forming a semiconductor channel;
applying a gate electrode that modulates current through the semiconductor channel, the gate electrode comprising two or more stacked layers, wherein the two or more stacked layers are composed of an oxide or a nitride; and
grafting a biorecognition layer to a top surface of the gate electrode.

12. The method of claim 11, wherein the oxide is a Group IV metal oxide, and wherein the nitride is a Group IV metal nitride.

13. The method of claim 11, wherein the two or more stacked layers of the gate electrode cause a reduction in desorption of the biorecognition layer into a solution.

14. The method of claim 11, wherein the biorecognition layer is an end-functionalized self-assembled monolayer.

15. The method of claim 11, further comprising:
grafting a biosensing agent to a top surface of the biorecognition layer.

16. The method of claim 11, further comprising:
forming a Silicon substrate in the semiconductor channel.

17. The method of claim 11, further comprising:
forming a Silicon Dioxide (SiO₂) substrate in the semiconductor channel for optical biosensing.

18. A semiconductor device, comprising:
a semiconductor channel, comprising a Silicon (Si) substrate;
a gate electrode comprising a first layer stacked on a second layer, wherein the first layer or the second layer is a Group IV metal oxides or a Group IV metal nitride; and
a biorecognition layer grafted to a top surface of the gate electrode.

19. The semiconductor device of claim 18, wherein the biorecognition layer is an end-functionalized self-assembled monolayer.

20. The semiconductor device of claim 18, wherein the biorecognition layer is an end-functionalized self-assembled monolayer.
